(19) United States
(12) Patent Application Publication
(10) Pub. No.: US 2003/0232871 A1
(43) Pub. Date: Dec. 18, 2003

(54) Crystalline parecoxib sodium

(76) Inventors: Ahmad Y. Sheikh, Des Plaines, IL (US); Thomas B. Borchardt, Kenosha, WI (US); Leonard J. Ferro, Highland Park, IL (US); Gerald D. Danzer, Racine, WI (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/387,173

(22) Filed: Mar. 12, 2003

Related U.S. Application Data
(60) Provisional application No. 60/364,567, filed on Mar. 15, 2002.
(60) Provisional application No. 60/417,987, filed on Oct. 11, 2002.

(51) Int. Cl. A61K 31/415; C07D 231/12

(52) U.S. Cl. 514/406; 548/377.1

(57) ABSTRACT

Parecoxib sodium is provided in a crystalline form that is substantially anhydrous and substantially nonsolvated. Various such anhydrous, nonsolvated crystal forms have been identified, including Forms A, B and E as described herein. Also provided is a parecoxib sodium drug substance wherein at least about 90% of the parecoxib sodium is in one or more anhydrous, nonsolvated crystal forms. Such a drug substance is a storage-stable intermediate that can be further processed, for example by dissolution or slurrying in an aqueous medium together with one or more parenterally acceptable excipients, followed by lyophilization of the resulting solution or slurry to provide a reconstitutable injectable composition suitable for therapeutic use.

This application claims priority of U.S. provisional application Serial No. 60/364,567 filed on Mar. 15, 2002, and U.S. provisional application Serial No. 60/417,987 filed on Oct. 11, 2002.

### FIELD OF THE INVENTION

The present invention is directed to parecoxib sodium crystal forms, to pharmaceutical compositions comprising such crystal forms, and to methods of using such compositions for treatment of cyclooxygenase-2 (COX-2) mediated disorders.

### BACKGROUND OF THE INVENTION

Nonsteroidal anti-inflammatory drugs (NSAIDs) are widely used to treat inflammation and pain, for example in arthritis and headache. Such drugs are effective but their long-term use can be limited by gastrointestinal side effects including dyspepsia and abdominal pain, and in severe cases by gastric or duodenal perforation and/or bleeding. Development of selective COX-2 inhibitory drugs has revolutionized treatment of inflammation and pain by combining the therapeutic effectiveness of traditional NSAIDs with a greatly improved gastrointestinal safety profile.

Inhibition of cyclooxygenase (COX) enzymes is believed to be at least the primary mechanism by which NSAIDs exert their characteristic anti-inflammatory, antipyretic and analgesic effects, through inhibition of prostaglandin synthesis. Conventional NSAIDs such as ketorolac, diclofenac, naproxen and salts thereof inhibit both the constitutively expressed COX-1 and the inflammation-associated or inducible COX-2 isoforms of cyclooxygenase at therapeutic doses. Inhibition of COX-1, which produces prostaglandins that are necessary for normal cell function, appears to account for certain adverse side effects that have been associated with use of conventional NSAIDs. By contrast, selective inhibition of COX-2 without substantial inhibition of COX-1 leads to anti-inflammatory, antipyretic, analgesic and other useful therapeutic effects while minimizing or eliminating such adverse side effects. Selective COX-2 inhibitory drugs have therefore represented a major advance in the art. These drugs are formulated in a variety of orally deliverable dosage forms.

Parenteral routes of administration, including subcutaneous, intramuscular and intravenous injection, offer numerous benefits over oral delivery in particular situations, for a wide variety of drugs. For example, parenteral administration of a drug typically results in attainment of a therapeutically effective blood serum concentration of the drug in a shorter time than is achievable by oral administration. This is especially true of intravenous injection, whereby the drug is placed directly in the bloodstream. Parenteral administration also results in more predictable blood serum concentrations of the drug, because losses in the gastrointestinal tract due to metabolism, binding to food and other causes are eliminated. For similar reasons, parenteral administration often permits dose reduction. Parenteral administration is generally the preferred method of drug delivery in emergency situations, and is also useful in treating subjects who are uncooperative, unconscious, or otherwise unable or unwilling to accept oral medication.

Relatively few NSAIDs are commercially available in injectable form. Non-selective NSAIDs such as ketorolac tromethamine salt that are available for parenteral use are effective analgesics but have been associated with side effects typical of such non-selective NSAIDs. These side effects have included upper gastrointestinal tract ulceration and bleeding, particularly in elderly subjects; reduced renal function, potentially leading to fluid retention and exacerbation of hypertension; and inhibition of platelet function, potentially predisposing the subject to increased bleeding, for example during surgery. Such side effects have seriously limited the use of parenteral formulations of non-selective NSAIDs.

Parecoxib, disclosed in U.S. Pat. No. 5,932,598 to Talley et al., is one of a class of water-soluble prodrugs of selective COX-2 inhibitory drugs. Parecoxib rapidly converts to the substantially water-insoluble selective COX-2 inhibitory drug valdecoxib following administration to a subject. Parecoxib also converts to valdecoxib upon exposure to water, for example upon dissolution in water. The high water solubility of parecoxib, particularly of salts of parecoxib such as the sodium salt, by comparison with most selective COX-2 inhibitory drugs such as celecoxib and valdecoxib, has led to interest in developing parecoxib for parenteral use. Parecoxib, having the structural formula (I) below, itself shows weak in vitro inhibitory activity against both COX-1 and COX-2, while valdecoxib (II) has strong inhibitory activity against COX-2 but is a weak inhibitor of COX-1.

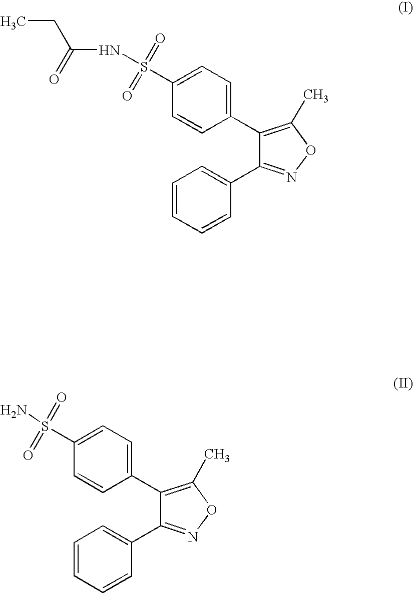

Parecoxib sodium has the structural formula (III) below.

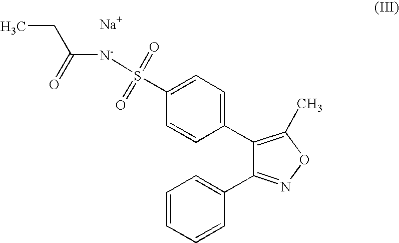

Above-cited U.S. Pat. No. 5,932,598 discloses parecoxib sodium in Example 18 thereof. Parecoxib can be synthesized by a procedure described in Examples 13 and 14 thereof, with substitution of the appropriate sulfonamide and anhydride.

There is a need for a stable crystalline form of parecoxib suitable as an active pharmaceutical ingredient (API), otherwise referred to herein as “drug substance”, that can be further processed to prepare a pharmaceutical composition for therapeutic use.

Crystalline structure of parecoxib sodium is not characterized in above-cited U.S. Pat. No. 5,932,598, except for disclosure of a melting point of 271.5-272.7° C. However, the process described therein involves a step of crystallization from ethanol, a step that is shown hereinbelow to generate an ethanol solvate. The melting point is not indicative of the solid state form as all crystal forms so far identified exhibit a similar melting point, in some cases following phase transition.

For provision of a commercial drug substance, anhydrous, nonsolvated crystal forms are generally preferred over solvates and hydrates, for various reasons including a tendency of such anhydrous, nonsolvated forms to exhibit enhanced physical stability. There thus exists a particular need in the art for an anhydrous, nonsolvated crystal form of parecoxib sodium, especially for such a crystal form having low hygroscopicity.

### SUMMARY OF THE INVENTION

There is now provided parecoxib sodium in a crystalline form that is substantially anhydrous and substantially nonsolvated. Various such anhydrous and nonsolvated crystal forms have now been identified.

In a first embodiment, Form A is provided. This crystal form of parecoxib sodium is anhydrous and nonsolvated and is characterized at least by a powder x-ray diffraction (PXRD) pattern having at least two 2θ values selected from the group consisting of 5.6, 9.6, 11.0 and 14.5 degrees.

All references herein to a 2θ value will be understood to be approximate and subject to normal measurement error depending on the apparatus and settings used, for example an error of ±0.2 degrees 2θ.

In a second embodiment, Form B is provided. This crystal form of parecoxib sodium is anhydrous and nonsolvated and is characterized at least by a PXRD pattern having at least two 2θ values selected from the group consisting of 4.2, 8.3, 12.4, 16.7, 17.5, 20.8 and 24.7 degrees.

In a third embodiment, Form E is provided. This crystal form of parecoxib is anhydrous and nonsolvated and is characterized at least by a PXRD pattern having at least two 2θ values selected from the group consisting of 8.8, 11.3, 15.6, 22.4, 23.5 and 26.4 degrees.

There is also provided a parecoxib sodium drug substance wherein at least about 90%, preferably at least about 95%, more preferably substantially all, of the parecoxib sodium is in one or more anhydrous, nonsolvated crystal forms as described above. Such a drug substance is a storage-stable intermediate that can be further processed, for example by dissolution or slurrying in an aqueous medium together with one or more parenterally acceptable excipients, followed by lyophilization of the resulting solution or slurry to provide a reconstitutable injectable composition suitable for therapeutic use.

Further provided is a method of treating a COX-2 mediated disorder in a subject, the method comprising administering to the subject a therapeutically effective amount of a pharmaceutical composition comprising such a parecoxib sodium drug substance and at least one pharmaceutically acceptable excipient.

Still further provided is a method of use of such a parecoxib sodium drug substance in manufacture of a medicament for treating a COX-2 mediated disorder.

### BRIEF DESCRIPTION OF THE DRAWINGS

FIG. 1 shows a PXRD pattern of parecoxib sodium Form A according to Example 4.

FIG. 2 shows a Fourier-transform infrared (FTIR) spectrum of parecoxib sodium Form A according to Example 5.

FIG. 3 shows a differential scanning calorimetry (DSC) thermogram of parecoxib sodium Form A according to Example 6.

FIG. 4 shows a moisture sorption profile at 25° C. for Form A according to Example 7.

FIG. 5 shows a PXRD pattern of parecoxib sodium Form B according to Example 4.

FIG. 6 shows an FTIR spectrum of parecoxib sodium Form B according to Example 5.

FIG. 7 shows a DSC thermogram of parecoxib sodium Form B according to Example 6.

FIG. 8 shows a moisture sorption profile at 25° C. for Form B according to Example 7.

FIG. 9 shows a PXRD pattern of parecoxib sodium Form E according to Example 4.

FIG. 10 shows an FTIR spectrum of parecoxib sodium Form E according to Example 5.

FIG. 11 shows a DSC thermogram of parecoxib sodium Form E according to Example 6.

FIG. 12 shows a moisture sorption profile at 25° C. for Form E according to Example 7.

### DETAILED DESCRIPTION OF THE INVENTION

It has been discovered that parecoxib sodium exists in an unexpected plurality of anhydrous, nonsolvated crystal forms. The discovery and characterization of these crystal forms, each of which exhibits advantages for manufacture, purification, storage and formulation of parecoxib sodium, constitute a major advance in the art by enhancing commercial feasibility of an important new therapeutic agent.

Numerous hydrates and solvates have also been observed. These tend to be unstable, gradually releasing water or solvent and converting to other solid state forms. It is possible that certain 2θ values indicated herein as characteristic of the PXRD pattern of forms A, B or E could also occur in a hydrate or solvate. However, the novel anhydrous, nonsolvated crystal forms of the present invention are readily distinguishable from such hydrates or solvates by the stability of their PXRD pattern in conditions wherein hydrates and solvates are unstable through release of water or solvent from the crystal lattice.

Form A

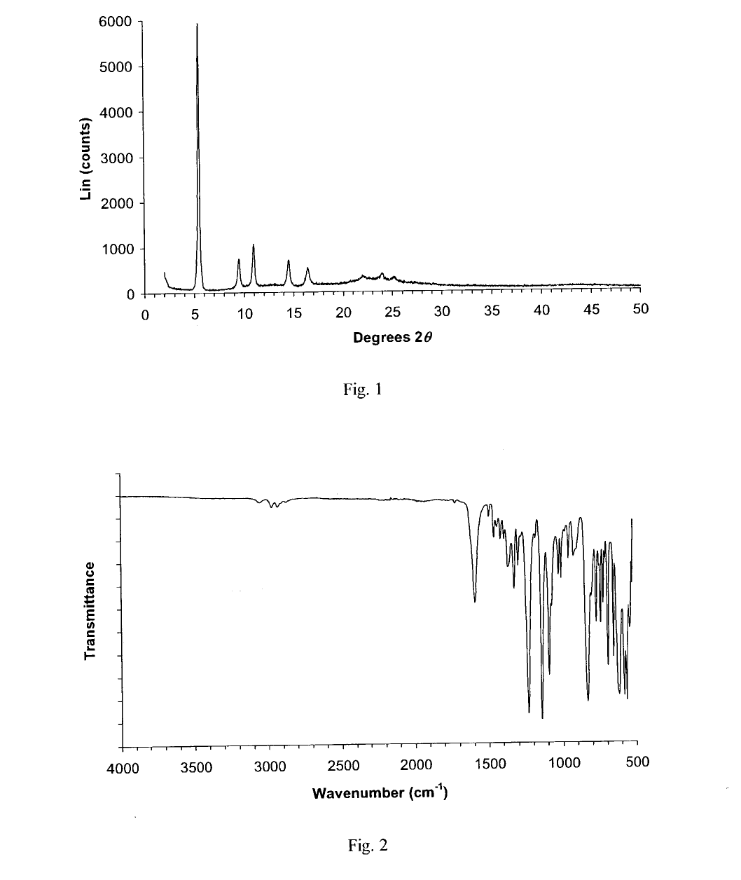

A first of the novel anhydrous, nonsolvated crystal forms exhibits a PXRD pattern having at least two 2θ values selected from the group consisting of 5.6, 9.6, 11.0 and 14.5 degrees, and is described herein as Form A. Alternatively or in addition, Form A can be characterized by a PXRD pattern having 2θ values substantially in accordance with Table 1 in Example 5 hereof. Alternatively or in addition, Form A can be characterized by a PXRD pattern substantially in accordance with FIG. 1.

Alternatively or in addition, Form A can be characterized by an FTIR spectrum substantially in accordance with FIG. 2.

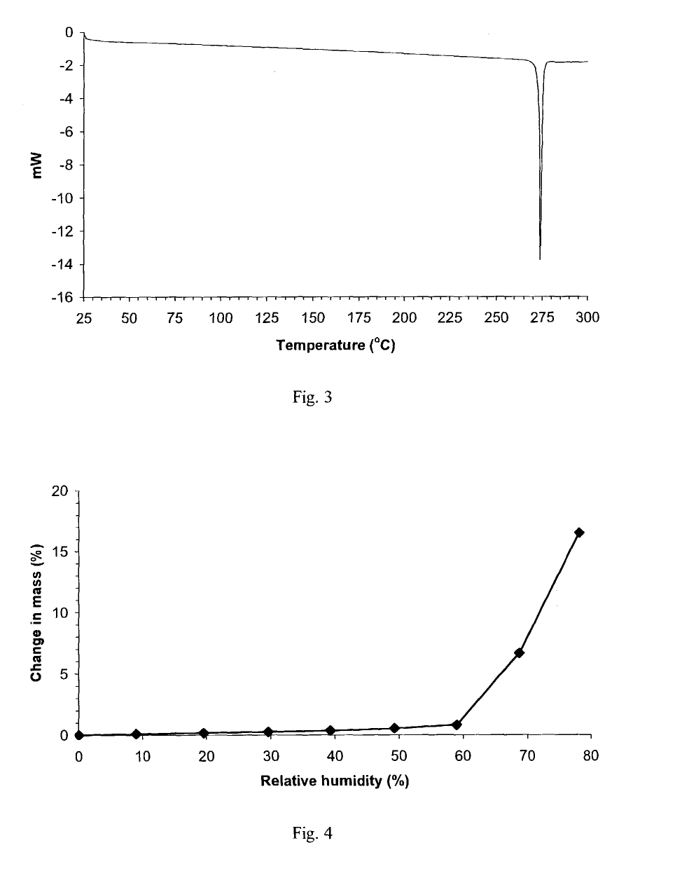

Alternatively or in addition, Form A can be characterized by a DSC thermogram substantially in accordance with FIG. 3.

In one preferred embodiment of the invention, a parecoxib sodium drug substance is provided wherein at least about 90%, more preferably at least about 95% and still more preferably substantially all of the parecoxib sodium is present as Form A. Such a drug substance is useful, in an amount of at least about 1 g, preferably at least about 10 g, more preferably at least about 100 g, and most preferably at least about 1 kg, for commercial-scale storage of parecoxib sodium and for further processing in manufacture of a formulated parecoxib sodium drug product suitable for therapeutic administration.

Form B

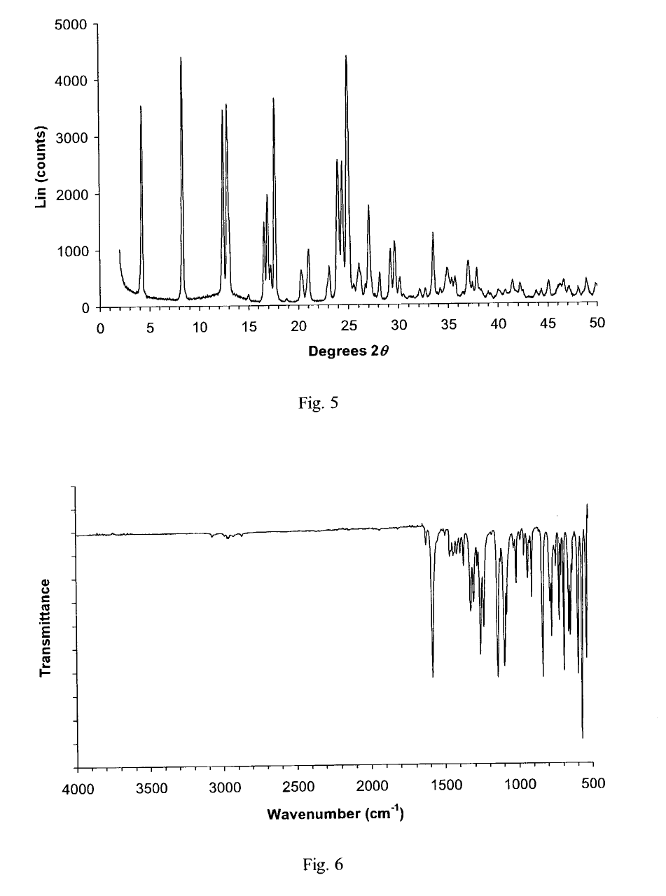

A second of the novel anhydrous, nonsolvated crystal forms exhibits a PXRD pattern having at least two 2θ values selected from the group consisting of 4.2, 8.3, 12.4, 16.7, 17.5, 20.8 and 24.7 degrees, and is described herein as Form B. Alternatively or in addition, Form B can be characterized by a PXRD pattern having 2θ values substantially in accordance with Table 2 in Example 5 hereof. Alternatively or in addition, Form B can be characterized by a PXRD pattern substantially in accordance with FIG. 5.

Alternatively or in addition, Form B can be characterized by an FTIR spectrum substantially in accordance with FIG. 6.

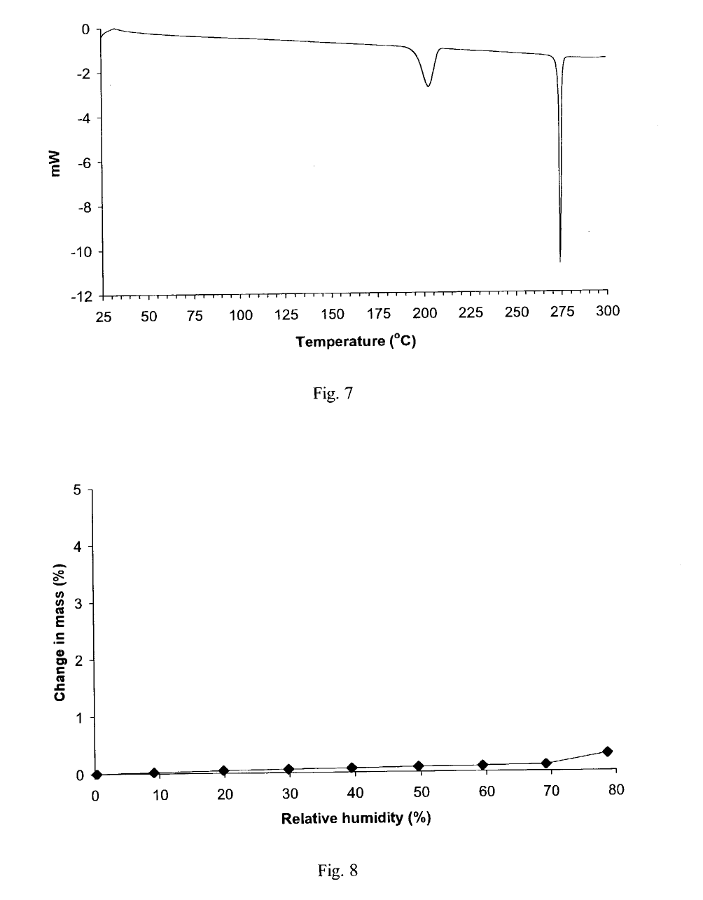

Alternatively or in addition, Form B can be characterized by a DSC thermogram substantially in accordance with FIG. 7.

In another preferred embodiment of the invention, a parecoxib sodium drug substance is provided wherein at least about 90%, more preferably at least about 95% and still more preferably substantially all of the parecoxib sodium is present as Form B.

Form E

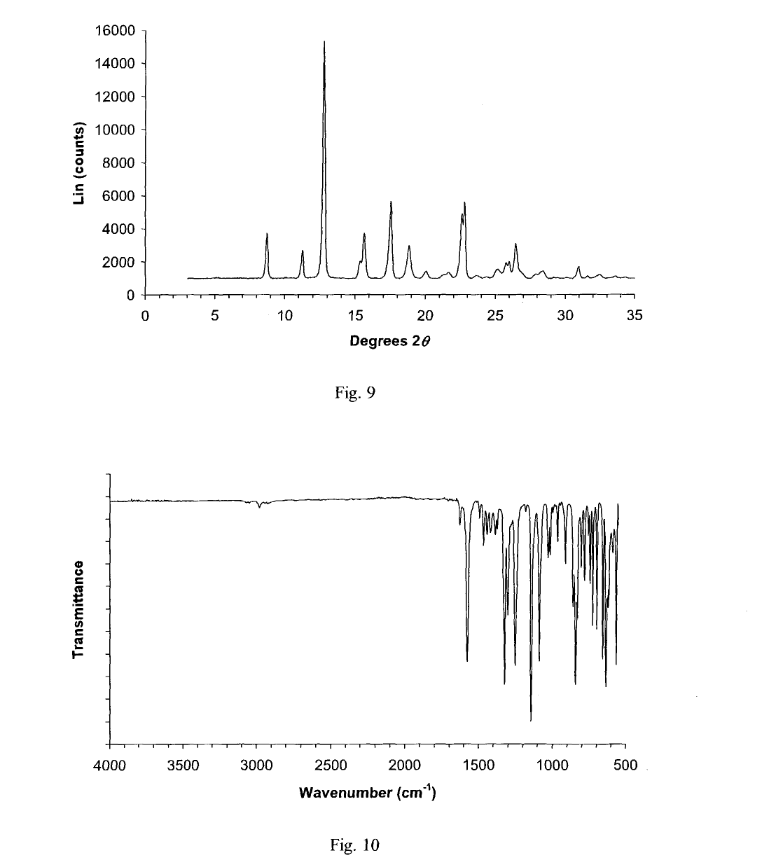

A third of the novel anhydrous, nonsolvated crystal forms exhibits a PXRD pattern having at least two 2θ values selected from the group consisting of 8.8, 11.3, 15.6, 22.4, 23.5 and 26.4 degrees, and is described herein as Form E. Alternatively or in addition, Form E can be characterized by a PXRD pattern having 2θ values substantially in accordance with Table 3 in Example 5 hereof. Alternatively or in addition, Form E can be characterized by a PXRD pattern substantially in accordance with FIG. 9.

Alternatively or in addition, Form E can be characterized by an FTIR spectrum substantially in accordance with FIG. 10.

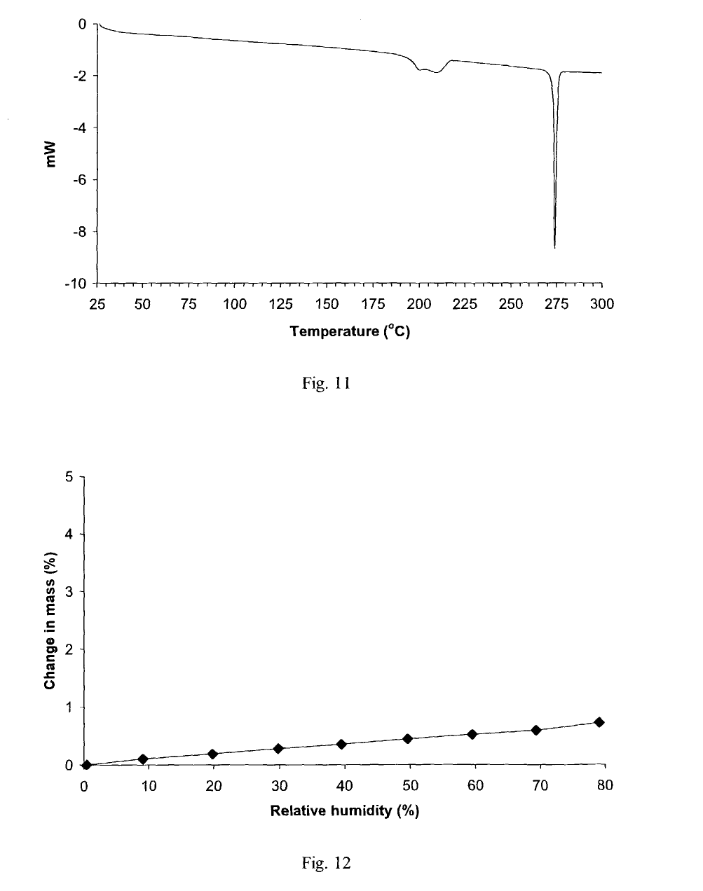

Alternatively or in addition, Form E can be characterized by a DSC thermogram substantially in accordance with FIG. 11.

In yet another preferred embodiment of the invention, a parecoxib sodium drug substance is provided wherein at least about 90%, more preferably at least about 95% and still more preferably substantially all of the parecoxib sodium is present as Form E.

Preparation of parecoxib sodium

Parecoxib sodium useful in preparation of any of the anhydrous, nonsolvated crystal forms or any of the parecoxib sodium drug substances described above can be prepared by any suitable process, including processes known per se. In one such process, synthesis of parecoxib sodium (III) involves five chemical steps starting with commercially available raw materials and is shown below in Scheme 1.

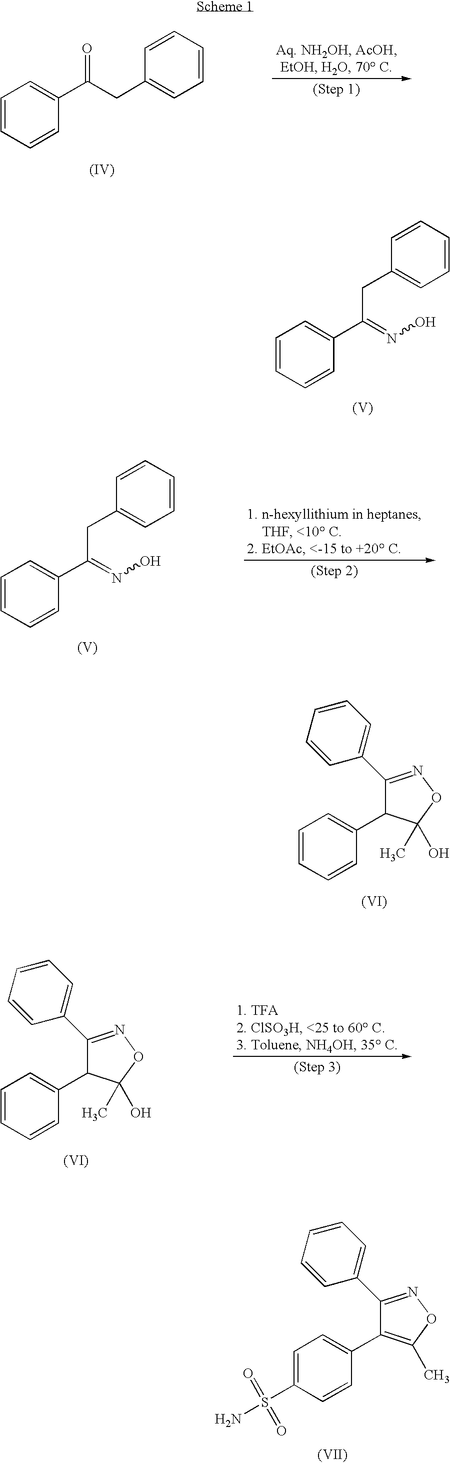

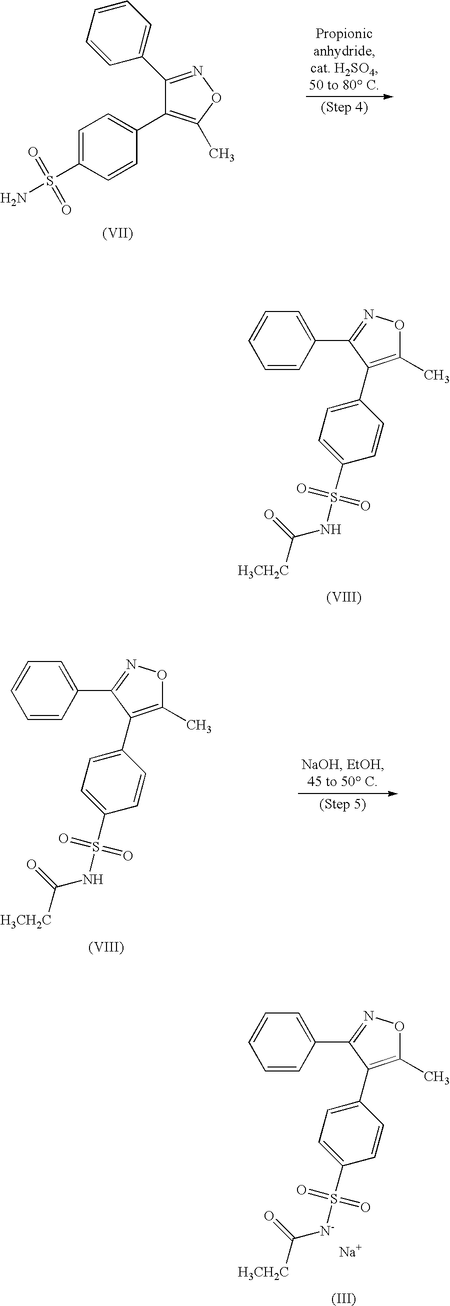

In the first step, a reaction vessel is charged with 210 kg deoxybenzoin (IV), 711 liters of ethanol, and 77 liters of 80% aqueous acetic acid. Alternatively, glacial acetic acid (63 liters) and water (16.5 liters) can be used. The mixture is heated to 70° C., and 71 liters of 50% aqueous hydroxylamine and 55 liters of water are added. The mixture is maintained at 70° C. for at least 1 hour. An in-process check is performed to ensure that the amount of unreacted deoxybenzoin (IV) is not more than 0.5%.

The mixture is cooled and maintained at 45° C. while water (266 liters) is added to crystallize the product. The mixture can be seeded if crystallization does not initiate. The temperature of the mixture is maintained at 45° C. for at least 1 hour and then water (816 liters) is slowly added to complete precipitation of product. The mixture is cooled to 20° C. and held at 20° C. for at least 1 hour.

The product is isolated, washed with a mixture of ethanol and water (at least 420 liters having a 1:2 ratio of ethanol to water) and then with water (at least 168 liters). The product is dried at up to 55° C. under vacuum, until residual water is not more than 0.5%, to give 1,2-diphenylethanone, oxime (V) in a typical yield of 223 kg (106% by weight).

In the second step, a reaction vessel is charged with 1,2-diphenylethanone, oxime (V) (93 kg) and tetrahydrofuran (THF, 620 liters). The solution was cooled, and n-hexyllithium (248 kg) is added while maintaining the temperature at or below 110° C. A minimum amount of heptanes is used to rinse the transfer lines, and the rinse is added to the reaction mixture.

After addition of n-hexyllithium is complete, the reaction mixture is cooled to −15° C. or below, and ethyl acetate (237 liters) is added. The reaction mixture is quenched by adding it to a solution of sodium chloride (41 kg) in water (474 liters) while maintaining the temperature at or below 15° C. The reaction vessel and transfer lines are rinsed with ethyl acetate (118 liters).

The layers are separated, and the organic phase is washed with a solution of sodium bicarbonate (28.4 kg) in water (474 liters). The organic phase is diluted with toluene (355 liters), and the mixture is distilled at atmospheric pressure until approximately two-thirds of the mass is removed. The hot solution is diluted with heptanes (1,300 liters), cooled to 5° C. and held at 5° C. for at least 1 hour. The precipitated product is isolated and washed with a mixture of heptanes and toluene (at least 110 liters having a 1:1 ratio of heptanes to toluene).

The product is dried under vacuum at up to 50° C. until the loss on drying (LOD) is not more than 0.5%, to give 4,5-dihydro-5-methyl-3,4-diphenyl-5-isoxazolol (VI) in a typical yield of 72 kg (77% by weight).

In the third step, a reaction vessel is charged with 4,5-dihydro-5-methyl-3,4diphenyl-5-isoxazolol (VI) (152 kg) and trifluoroacetic acid (TFA, 116 liters). The mixture is cooled and chlorosulfonic acid (705 kg) is added while maintaining the temperature of the reaction mixture below 25° C.

After the addition is complete, the mixture is slowly heated to 60° C. and held at 60° C. for at least 1 hour. The reaction mixture is cooled and quenched by adding it to a mixture of water (456 liters) and toluene (570 liters) that is maintained below 25° C. during this addition. The reaction vessel and transfer lines are rinsed with a mixture of water (152 liters) and toluene (61 liters). The layers are separated, and the organic phase is washed with water (220 liters).

The organic phase is treated with aqueous ammonium hydroxide (190 liters), and the mixture is heated to 35° C. and held at 35° C. for at least 30 minutes. An in-process check is performed to ensure that pH of the aqueous phase is not less than 9.

Isopropyl alcohol (729 liters) is added, and the mixture is held at 35° C. for at least 1 hour. The mixture is cooled to 20° C. and held at 20° C. for at least 1 hour. The precipitated product is isolated and washed with isopropyl alcohol (304 liters) and then with water (at least 101 liters).

The crude product is dissolved in hot methanol (709 liters). The solution is filtered to remove particulates and diluted with additional methanol (355 liters) and water (274 liters). The mixture is heated to 70° C. to dissolve the solid and then slowly cooled to initiate crystallization of the product. The mixture can be seeded if crystallization does not initiate by the time 45° C. is reached. Once crystallization occurs, the mixture is stirred at 50° C. for at least 1 hour and then slowly cooled to 5-10° C. and held at that temperature for at least 1 hour. The product is isolated and washed with a mixture of methanol and water (at least 95 liters having a 3:1 ratio of methanol to water). Alternatively, the product can be purified by recrystallization from a mixture of ethanol (1,300 liters) and water (68 liters) using the same procedure described above.

The product is dried under vacuum at temperatures up to 100° C. until the amount of residual solvents by LOD or gas chromatography is not more than 0.5%, to give 4-(5-methyl-3-phenyl-4-isoxazolyl)benzenesulfonamide (VII) in a typical yield of 103 kg (62% by weight).

In the fourth step, a reaction vessel is charged with 4-(5-methyl-3-phenyl-4-isoxazolyl)benzenesulfonamide (VII) (21 kg) and propionic anhydride (86 kg). The resulting suspension is warmed to 50° C., and sulfuric acid (21 ml) is added. The reaction mixture is warmed to 80° C. and held for at least 30 minutes.

The mixture is slowly cooled to 50° C. to initiate crystallization of the product. The mixture is held at 50° C. for at least 30 minutes after crystallization is initiated. The mixture can be seeded if crystallization does not initiate at 50° C. The mixture is slowly cooled to 0° C. and held at 0° C. for at least 1 hour to complete the crystallization.

The product was isolated, washed with methyl tert-butyl ether (80 liters), and partially dried on the filter until an in-process check indicates that LOD is not more than 5%, to give n-[[4-(5-methyl-3-phenyl-4-isoxazolyl)phenyl]sulfonyl]propanamide (VIII) as a wet cake that is carried directly into the fifth step without further purification or drying.

In the fifth step, the wet cake obtained in the fourth step is dissolved in absolute ethanol (12.6 kg/kg of (VIII) on a dry weight basis) at 45° C., and the mixture is filtered to remove particulates.

A solution of sodium hydroxide (approximately 5% by weight) in absolute ethanol is prepared in a separate reaction vessel, and the molarity of the solution is determined by titration. The calculated amount of the sodium hydroxide solution is added through an in-line filter to the solution of (VIII) in ethanol, and the mixture is maintained at 45° C. and seeded to initiate crystallization.

After seeding, the mixture is warmed to 50° C., held for at least 30 minutes, and then cooled to 0° C. to complete the crystallization. The mixture is stirred at 0° C. for at least 30 minutes, and the product is isolated and washed with cold absolute ethanol (at least 88 kg).

Finally, the product is dried under vacuum at up to 135° C. to give parecoxib sodium (III) in a typical yield of 17.2 kg (82% by weight).

It will be understood that the above process description is provided for illustrative purposes. Variations of the above process, including in process conditions and in scale, will be readily made by one of skill in the art without departing from the present invention.

Preparation of parecoxib sodium Forms A B and E

Surprisingly, it has been discovered that during the fifth step of the above described process, slight changes in drying conditions produce a variety of anhydrous, solvated and hydrated crystal forms. Typically, at least a portion of the parecoxib sodium produced is in the form of an ethanol solvate. Ethanol solvates of parecoxib sodium can be produced having different stoichiometries, i.e., higher and lower ethanol solvates, that are directly related to drying efficiency.

Regardless of the crystal form of parecoxib sodium obtained in the fifth step, however, if temperature is increased to about 210° C. during or following drying, the parecoxib sodium converts to Form A. On cooling, the parecoxib sodium remains as Form A.

Accordingly, a first process for preparation of Form A parecoxib sodium is provided, comprising a step of heating a crystal form of parecoxib sodium other than Form A to a temperature from about 210° C. to the melting point of parecoxib sodium, for a period sufficient to convert the parecoxib sodium to Form A, and cooling the resulting Form A parecoxib sodium to ambient temperature.

It has further been discovered that a mixture of Form A and ethanol solvate of parecoxib sodium can be converted to substantially pure Form A by heating the mixture at ambient pressure for about 3 hours at about 150° C.

Accordingly, a second process for preparation of Form A parecoxib sodium is provided, comprising a step of heating an ethanol solvate of parecoxib sodium in presence of Form A parecoxib sodium to a temperature from about 150° C. to the melting point of parecoxib sodium, for a period sufficient to convert the ethanol solvate to Form A, and cooling the resulting Form A parecoxib sodium to ambient temperature.

It has still further been discovered that an amorphous form of parecoxib sodium, which can be prepared by dissolution of any solid state form of parecoxib sodium in water followed by lyophilization, is converted to Form A when heated to about 125° C. to about 130° C. in absence of moisture.

Accordingly, a third process for preparation of Form A parecoxib sodium is provided, comprising a step of heating amorphous or lyophilized parecoxib sodium in substantial absence of moisture to a temperature from about 125° C. to the melting point of parecoxib sodium, for a period sufficient to convert the amorphous or lyophilized parecoxib sodium to Form A, and cooling the resulting Form A parecoxib sodium to ambient temperature.

A process for preparation of a parecoxib sodium drug substance having at least about 90% Form A comprises the steps of (a) crystallizing parecoxib sodium from a crystallizing solvent (e.g., ethanol) to produce a crystalline form of parecoxib sodium, and (b) heating the resulting crystalline parecoxib sodium at a temperature of about 110° C. to about 230° C. to produce the desired parecoxib sodium drug substance.

At relative humidity (RH) levels higher than about 60% RH, Form A converts over time to a hydrated crystalline form. Complete conversion to a hydrate occurs, for example, following exposure of Form A to about 75% RH for about 3 to about 7 days. It has been found that when such a hydrate is dried at ambient temperature, for example by drying over an efficient desiccant such as P₂O₅, the solid state form does not revert to Form A but instead becomes Form B.

Accordingly, a process for preparation of Form B parecoxib sodium is provided, comprising a step of drying a hydrated crystalline form of parecoxib sodium over a desiccant at a temperature below that giving rise to Form A, to produce Form B parecoxib sodium.

Form E parecoxib sodium can be prepared by recrystallizing an ethanol solvate of parecoxib sodium from heptane to produce Form E crystals.

Properties of parecoxib sodium Forms A, B and E

Moisture sorption isotherms for Forms A, B and E at ambient temperature are shown in FIGS. 4, 8 and 12 respectively. Form A sorbs less than 1% moisture below about 60% RH but above about 60% RH has greater tendency to sorb water and even to deliquesce. Forms B and E are less hygroscopic than Form A, showing little tendency to sorb water even at up to about 80% RH.

The lower hygroscopicity of Forms B and E by comparison with Form A can be reconciled by reference to relative thermodynamic stability of these solid state forms. As shown in the energy/temperature diagram of FIG. 17, Form A is higher in energy than Forms B and E, which are similar to each other. It is believed, without being bound by, theory, that Forms B and E are less hygroscopic than Form A because they represent lower energy, i.e., more thermodynamically stable, states.

The relative ease with which Form A can be prepared from other solid state forms of parecoxib sodium at a commercial scale, for example by a heating and cooling process, is unexpected and confers a major commercial advantage to Form A. Once prepared, Form A exhibits a high degree of stability and in this respect provides a benefit over hydrates and solvates, for example the ethanol solvate believed to result from the process suggested by above-cited U.S. Pat. No. 5,932,598. Existence of various hydrates and solvates at different stoichiometries leads to product variation, which is overcome by the present invention. Where lower hygroscopicity is desired, Form B and Form E have an advantage in this regard over Form A.

Utility of parecoxib sodium Forms A, B and E

As previously noted, the new crystalline forms of parecoxib sodium provided by the present invention are especially suitable for use as a drug substance or API that can be stored until ready for downstream processing to prepare a pharmaceutical composition. These forms can, if desired, be incorporated as such, together with one or more pharmaceutically acceptable excipients, in a solid state formulation such as a tablet or capsule for oral administration or a gel or patch for topical administration. If necessary particle size of these crystalline forms can be reduced or rendered more uniform by milling or grinding or other physical means, prior to formulation preparation.

Alternatively, the new crystalline forms can be converted to a non-crystalline form, for example a solution or an amorphous form, in preparation of a pharmaceutical composition. For example, the new crystalline forms can be regarded as stable process intermediates.

In one embodiment of the present invention there is provided a process for preparing a pharmaceutical composition useful in treatment of a COX-2 mediated disorder, the process comprising a step of dissolving in an aqueous medium a parecoxib sodium drug substance wherein at least about 90% of the parecoxib sodium is in one or more of Forms A, B and E, together with at least one pharmaceutically acceptable excipient, to form a solution.

Such a solution can be a ready-to-use injectable composition. Alternatively, such a solution can be subjected to a further step of lyophilization to provide a solid particulate pharmaceutical composition comprising amorphous parecoxib sodium. Such a composition can be reconstituted by addition of a parenterally acceptable aqueous diluent to form an injectable solution of parecoxib sodium. The term “solution” as applied to a material to be lyophilized will be understood to embrace a slurry as well as a true solution.

According to the present embodiment, it is preferred that at least about 90%, more preferably at least about 95%, of the drug substance to be dissolved in the aqueous medium prior to formation of the pharmaceutical composition is Form A or Form B or Form E. Most preferably, such a drug substance is substantially phase pure Form A, Form B or Form E.

Therapeutic Method of Use

A drug substance of the invention, upon conversion to or incorporation in a pharmaceutical composition as indicated above, is useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects, especially when systemically administered, than compositions of conventional NSAIDs that lack selectivity for COX-2 over COX-1. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendinitis, bursitis, allergic neuritis, cytomegalovirus infectivity, apoptosis including HIV-induced apoptosis, lumbago, liver disease including hepatitis, skin-related conditions such as psoriasis, eczema, acne, bums, dermatitis and ultraviolet radiation damage including sunburn, and post-operative inflammation.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic diseases, such as retinitis, conjunctivitis, retinopathies, uveitis, ocular photophobia, and of acute injury to the eye tissue.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are used in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemangiomas, angiofibroma of the nasopharynx and avascular necrosis of bone; and disorders of the female reproductive system such as endometriosis.

Such compositions are useful in the treatment of pre-cancerous diseases, such as actinic keratosis.

Such compositions are useful in prevention, treatment and inhibition of benign and malignant tumors and neoplasia including neoplasia in metastasis, for example in colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in patients at risk of FAP.

More particularly, the compositions can be used in treatment, prevention and inhibition of acral lentiginous melanoma, actinic keratoses, adenocarcinoma, adenoid cystic carcinoma, adenoma, adenosarcoma, adenosquamous carcinoma, astrocytic tumors, bartholin gland carcinoma, basal cell carcinoma, breast cancer, bronchial gland carcinoma, capillary hemangioma, carcinoids, carcinosarcoma, cavernous hemangioma, cholangiocarcinoma, chondrosarcoma, chorioid plexus papilloma or carcinoma, clear cell carcinoma, cutaneous T-cell lymphoma (mycosis fungoides), cystadenoma, displastic nevi, endodermal sinus tumor, endometrial hyperplasia, endometrial stromal sarcoma, endometrioid adenocarcinoma, ependymoma, epithelioid angiomatosis, Ewing's sarcoma, fibrolamellar sarcoma, focal nodular hyperplasia, gastrinoma, germ cell tumors, glioblastoma, glucagonoma, hemangioblastoma, hemangioendothelioma, hemangioma, hepatic adenoma, hepatic adenomatosis, hepatocellular carcinoma, insulinoma, intraepithelial neoplasia, interepithelial squamous cell neoplasia, invasive squamous cell carcinoma, Kaposi's sarcoma, large cell carcinoma, leiomyosarcoma, lentigo-maligna melanoma, malignant melanoma, malignant mesothelial tumors, medulloblastoma, medulloepithelioma, melanoma, meningioma, mesothelioma, mucoepidermoid carcinoma, neuroblastoma, neuroepithelial adenocarcinoma, nodular melanoma, oat cell carcinoma, oligodendroglioma, osteosarcoma, papillary serous adenocarcinoma, pineal tumors, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinoma, somatostatin-secreting tumor, squamous carcinoma, squamous cell carcinoma, submesothelial carcinoma, superficial spreading melanoma, undifferentiated carcinoma, uveal melanoma, verrucous carcinoma, vipoma, well differentiated carcinoma and Wilm's tumor.

Such compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for compositions of the invention are for treatment of rheumatoid arthritis and osteoarthritis, for pain management generally (particularly post-oral surgery pain, post-general surgery pain, post-orthopedic surgery pain, and acute flares of osteoarthritis), for prevention and treatment of headache and migraine, for treatment of Alzheimer's disease, and for colon cancer chemoprevention.

Administration can be by any route, including parenteral, oral, rectal, pulmonary, nasal, otic and topical. Topical application of a parecoxib sodium composition prepared from one or more of Forms A, B and E can be especially useful in treatment of any kind of dermal disorder having an inflammatory component, whether malignant, non-malignant or pre-malignant, including scar formation and ketosis, and also including burns and solar damage, for example sunburn, wrinkles, etc. Such compositions can be used to treat inflammation resulting from a variety of skin injuries including without limitation those caused by viral diseases including herpes infections (e.g., cold sores, genital herpes), shingles and chicken pox. Other lesions or injuries to the skin that can be treated with such compositions include pressure sores (decubitus ulcers), hyperproliferative activity in the epidermis, miliria, psoriasis, eczema, acne, dermatitis, itching, warts and rosacea. Such compositions can also facilitate healing processes after surgical procedures, including cosmetic procedures such as chemical peels, laser treatment, dermabrasion, facelifts, eyelid surgery, etc.

Besides being useful for human treatment, compositions of the invention are also useful for veterinary treatment of companion animals, exotic animals, farm animals, and the like, particularly mammals including rodents. More particularly, compositions of the invention are useful for veterinary treatment of COX-2 mediated disorders in horses, dogs and cats.

The present compositions can be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, ε-acetamidocaproic acid, acetaminophen, acetaminosalol, acetanilide, acetylsalicylsalicylic acid, S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, aspirin, balsalazide, bendazac, benorylate, benoxaprofen, benzpiperylon, benzydamine, benzylmorphine, berberine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butorphanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, dipyrocetyl, dipyrone, ditazol, droxicam, emorfazone, enfenamic acid, epirizole, eptazocine, etanercept, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, infliximab, interleukin-10, isofezolac, isoladol, isomethadone, isonixin, isoxepac, isoxicam, ketobemidone, ketoprofen, ketorolac, p-lactophenetide, lefetamine, levorphanol, lexipafant, lofentanil, lonazolac, lornoxicam, loxoprofen, lysine acetylsalicylate, magnesium acetylsalicylate, meclofenamic acid, mefenamic acid, meloxicam, meperidine, meptazinol, mesalamine, metazocine, methadone, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalate, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen, ziconotide and zomepirac (see The Merck Index, 13th Edition (2001), Therapeutic Category and Biological Activity Index, lists therein headed “Analgesic”, “Anti-inflammatory” and “Antipyretic”).

Particularly preferred combination therapies comprise use of a composition of the invention with an opioid compound, more particularly where the opioid compound is codeine, meperidine, morphine or a derivative thereof.

The compound to be administered in combination with the composition of the invention can be formulated separately therefrom, and administered by any suitable route, including orally, rectally, parenterally or topically to the skin or elsewhere. Alternatively, the compound to be administered in combination with the present composition can be coformulated therewith as a coated sheet composition.

In an embodiment of the invention, particularly where the COX-2 mediated condition is headache or migraine, the present composition is administered in combination therapy with a vasomodulator, preferably a xanthine derivative having vasomodulatory effect, more preferably an alkylxanthine compound.

Combination therapies wherein an alkylxanthine compound is co-administered with a composition as provided herein are embraced by the present embodiment of the invention whether or not the alkylxanthine is a vasomodulator and whether or not the therapeutic effectiveness of the combination is to any degree attributable to a vasomodulatory effect. The term “alkylxanthine” herein embraces xanthine derivatives having one or more C₁₄ alkyl, preferably methyl, substituents, and pharmaceutically acceptable salts of such xanthine derivatives. Dimethylxanthines and trimethylxanthines, including caffeine, theobromine and theophylline, are especially preferred. Most preferably, the alkylxanthine compound is caffeine.

The vasomodulator or alkylxanthine component of the combination therapy can be administered in any suitable dosage form by any suitable route, including orally, rectally, parenterally or topically to the skin or elsewhere. The vasomodulator or alkylxanthine can optionally be coformulated with the present composition in a single transdermal dosage form. Thus a transdermal composition of the invention optionally comprises both valdecoxib or a prodrug thereof or a salt thereof and a vasomodulator or alkylxanthine such as caffeine, in total and relative amounts that are therapeutically effective.

### EXAMPLES

The following examples contain detailed descriptions that illustrate the invention without in any way restricting its scope. All percentages are by weight unless otherwise indicated. The parecoxib sodium starting material used in each of the following Examples was prepared in accordance with Scheme 1 above.

### Example 1

### Preparation of Form A

Parecoxib sodium Form A was prepared by each of the following methods.

1. An aqueous solution of parecoxib sodium was lyophilized. The resulting amorphous parecoxib sodium was placed in a DSC pan in absence of moisture and was subjected to temperature increase at a rate of 10° C./minute. Crystallization of the parecoxib sodium occurred as an exothermic event at about 125-130° C. The crystals were confirmed to be Form A by one or more of PXRD, FTIR, DSC and moisture sorption as described below.

2. A mixture of Form A and an ethanol solvate of parecoxib sodium, in a total amount of 10 g, was placed in an oven at 150° C. at ambient pressure for 3 hours. The resulting solid was cooled in a desiccator jar containing Drierite desiccant and was confirmed to be Form A by one or more of PXRD, FTIR, DSC and moisture sorption as described below.

3. Form E parecoxib sodium was found to convert to Form A as a solid-state transition observed by DSC as a broad-band endothermic event at about 210° C. Form A was confirmed by one or more of PXRD, FTIR, DSC and moisture sorption as described below.

Form A was characterized by PXRD, FTIR, DSC and moisture sorption data as shown in FIGS. 1-4 respectively.

### Example 2

### Preparation of Form B

Parecoxib sodium Form B was prepared by each of the following methods.

1. Parecoxib sodium Form A was exposed to about 75% RH for several days to produce a hydrated crystalline form. This hydrated form was then dried over a desiccant. The resulting solid was confirmed to be Form B by one or more of PXRD, FTIR, DSC and moisture sorption as described below.

2. An ethanol solvate of parecoxib sodium was prepared by recrystallizing 11.5 g of parecoxib sodium in 100 ml ethanol by heating to boiling on a hot plate with magnetic stirring, followed by ambient cooling to room temperature. Separately, about 1 g of Form B seed crystals was added to 450 ml heptane. The freshly prepared ethanol solvate was collected by vacuum filtration and immediately transferred into the heptane suspension containing Form B seed crystals. The resulting suspension was heated to reflux for 4 hours with vigorous magnetic stirring. Crystals were collected by vacuum filtration and dried at 40° C. under house vacuum overnight, and were confirmed to be Form B by one or more of PXRD, FTIR, DSC and moisture sorption as described below.

Form B was characterized by PXRD, FTIR, DSC and moisture sorption data as shown in FIGS. 5-8 respectively.

### Example 3

### Preparation of Form E

Parecoxib sodium Form E was prepared as follows. An ethanol solvate crystal form of parecoxib sodium, prepared by method 2 of Example 2, was transferred to 450 ml heptane, without seeding. The resulting suspension was heated to reflux for 4 hours with vigorous magnetic stirring. Crystals were collected by vacuum filtration and dried at 40° C. under house vacuum overnight, and were confirmed to be Form E by one or more of PXRD, FTIR, DSC and moisture sorption as described below.

Form E was characterized by PXRD, FTIR, DSC and moisture sorption data as shown in FIGS. 9-12 respectively.

### Example 4

### PXRD

Powder x-ray diffraction (PXRD) data were collected with a Siemens D5000 or an Inel Multipurpose Diffractometer using Cu-Kα radiation at a voltage of 30 kV and a current of 30 mA. The Inel was equipped with a position sensitive detector that allowed for acquisition of all diffraction data simultaneously. The diffractometer was calibrated against silicon and mica reference standards along with the direct beam. Capillary measurements were performed in 1 mm sealed glass capillaries mounted on a goniometer head within a capillary furnace. For the capillary measurements, the diffractometer was calibrated against silicon and the direct beam.

The diffraction patterns for parecoxib sodium Forms A, B and E are shown in FIGS. 1, 5 and 9 respectively, and diffraction peaks for each form are listed in Tables 1, 2 and 3 respectively.

TABLE 1
PXRD Peaks for Form A

| d-Spacing | Angle 2θ | Intensity |
|---|---|---|
| (Å) | (±0.2) | (%) |
| 15.7 | 5.6 | 100.0 |
| 9.3 | 9.6 | 10.3 |
| 8.0 | 11.0 | 12.7 |
| 6.1 | 14.5 | 6.0 |
| 5.4 | 16.5 | 6.5 |
| 4.0 | 22.0 | 1.3 |
| 3.7 | 24.0 | 3.7 |
| 3.5 | 25.3 | 2.5 |

TABLE 2
PXRD Peaks for Form B

| d-Spacing | Angle 2θ | Intensity |
|---|---|---|
| (Å) | (±0.2) | (%) |
| 20.9 | 4.2 | 74.3 |
| 10.6 | 8.3 | 81.1 |
| 7.2 | 12.3 | 39.3 |
| 7.2 | 12.4 | 22.7 |
| 6.9 | 12.8 | 100.0 |
| 6.8 | 13.0 | 8.0 |
| 6.0 | 14.8 | 1.0 |
| 5.4 | 16.4 | 22.0 |
| 5.3 | 16.7 | 14.6 |
| 5.2 | 16.1 | 9.7 |
| 5.1 | 17.5 | 32.4 |
| 4.7 | 18.7 | 0.9 |
| 4.4 | 20.1 | 8.6 |
| 4.3 | 20.6 | 3.0 |
| 4.3 | 20.8 | 8.1 |
| 3.9 | 22.7 | 4.0 |
| 3.9 | 22.9 | 2.6 |
| 3.7 | 23.8 | 21.4 |
| 3.7 | 24.2 | 23.4 |
| 3.6 | 24.7 | 74.9 |

TABLE 3
PXRD Peaks for Form E

| d-Spacing | Angle 2θ | Intensity |
|---|---|---|
| (Å) | (±0.2) | (%) |
| 10.0 | 8.8 | 26.2 |
| 7.9 | 11.3 | 12.7 |
| 6.9 | 12.8 | 100.0 |
| 5.8 | 15.3 | 5.4 |
| 5.7 | 15.6 | 22.4 |
| 5.1 | 17.4 | 45.0 |
| 4.7 | 18.7 | 25.7 |
| 4.5 | 19.9 | 4.1 |
| 4.2 | 21.1 | 3.8 |
| 4.1 | 21.5 | 3.2 |
| 4.0 | 22.4 | 40.8 |
| 3.9 | 22.7 | 25.5 |
| 3.8 | 23.5 | 11.5 |
| 3.7 | 24.2 | 0.9 |
| 3.6 | 25.0 | 5.8 |
| 3.5 | 25.7 | 9.6 |
| 3.4 | 25.9 | 3.9 |
| 3.4 | 26.4 | 35.2 |
| 3.3 | 26.8 | 7.4 |
| 3.2 | 27.8 | 2.6 |

### Example 5

### FTIR Spectroscopy

Fourier-transform infrared (FTIR) spectra were recorded with a Nicolet Nexus 670 FT-IR spectrophotometer. Samples were scanned using a Nicolet SMART DuraSamplIR attenuated total reflectance (ATR) accessory. Samples were scanned at a resolution of 4 cm⁻¹ averaging a total of 64 scans from 4000 to 400 cm⁻¹.

FTIR spectra of parecoxib sodium Forms A, B and E from 4000 to 500 cm⁻¹ are shown in FIGS. 2, 6 and 10 respectively.

### Example 6

### DSC

Differential scanning calorimetry (DSC) data were collected with a Mettler-Toledo DSC 821. The temperature and enthalpy were calibrated with indium and zinc reference standards. Samples were analyzed in either sealed or pinpricked 40 μl aluminum pans from 25° C. to 300° C. The heating rate was 10° C./minute and the nitrogen purge rate was 50 ml/minute.

DSC thermograms for parecoxib sodium Forms A, B and E are shown in FIGS. 3, 7 and 11 respectively.

Form A displayed a single melting endotherm with an onset at about 273.1° C. (ΔHₜ=23.8 kJ/mole). Form B displayed an endotherm with an onset at about 195.9° C. (ΔHₜ=20.71 kJ/mole) representing transition to Form A, followed by a sharp melting endotherm for Form A at 273.7° C. Form E displayed a broad endotherm with an onset at about 206.6° C. (ΔHₜ=18.35 kJ/mole) representing transition to Form A, followed by a sharp melting endotherm for Form A at 273.2° C. The transitions for Forms B and E to Form A prior to melting were verified to be solid-solid transitions by hot-stage microscopy.

Based on the Heat of Transition Rule both Forms B and E are believed to be enantiotropically related to Form A, meaning there is a change in the stability relationship between the forms around a transition temperature Tₜ. Determination of Tₜ for Forms B and E with respect to Form A was performed by the use of eutectic melting data.

Eutectics were formed between a reference compound (RC) and each of Forms A, B and E of parecoxib sodium. Subsequently heat of fusion data were used to derive the free energy difference between the crystal forms at the eutectic temperature (equation I):

xₑⱼ(Gⱼ−Gᵢ₎Tₑᵢ=ΔHₘₑⱼ(Tₑᵢ−Tₑⱼ)/Tₑ−ΔCₚᵢⱼ[Tₑᵢ−Tₑⱼ−Tₑᵢ ln(Tₑᵢ/Tₑⱼ)]+Tₑᵢ{Xₑⱼ ln(Xₑⱼ/Xₑᵢ)+(1−Xₑⱼ)ln[(1−Xₑⱼ)/(1−Xₑᵢ)]} (equation I)

wherein xₑⱼ and xₑᵢ are the mole fraction of crystal forms j and i respectively in the eutectic; (Gⱼ−Gᵢ) is the free energy difference between crystal forms i and j at Tₑᵢ; ΔHₘₑⱼ and ΔHₘₑᵢ are the enthalpy of eutectic melting of crystal forms j and i respectively; Tₑᵢ and Tₑⱼ are the temperatures of eutectic melting of crystal forms i and j respectively; ΔCₚᵢⱼ is the heat capacity change across the eutectic melt; and R is the ideal gas constant.

The eutectic melting data for Forms A, B and E with selected reference compounds are given in Table 4.

TABLE 4
Eutectic melting data for Forms A, B and E

| | Form A | Form B | Form E |
|---|---|---|---|
| melting point, ° C. | 274-276 | Phase Conversion | Phase Conversion |
| RC is phenacetin | | | |
| χₑ | 0.25 | 0.25 | 0.25 |
| Tₑ, ° C. (mean) | 118.2 | 124.7 | 124.7 |
| ΔHₘₑ, kJ/mole | 24.64 | 25.99 | 27.08 |
| RC is benzanilide | | | |
| χₑ | 0.17 | 0.18 | 0.18 |
| Tₑ, ° C. (mean) | 155.6 | 156.6 | 156.2 |
| ΔHₘₑ, kJ/mole | 28.32 | 31.95 | 31.42 |
| RC is salophen | | | |
| χₑ | 0.42 | 0.42 | 0.42 |
| Tₑ, ° C. (mean) | 171.7 | 170.1 | 170.1 |
| ΔHₘₑ, kJ/mole | 25.82 | 36.83 | 34.62 |

The eutectic melting data confirm an enantiotropic relationship between Forms A and either B or E. Other thermodynamic parameters derived from plots of ΔG−T (ΔS) and ΔG/T−1/T (ΔH) are given in Table 5. The AH for Form E/Form A and Form B/Form A pairs from solution calorimetry measurements is also provided in Table 5 for comparison.

TABLE 5
Thermodynamic parameters

| Forms/Transition | ΔH (kJ/mole) | ΔS (J/mole/K) | Tₜ (° C.) |
|---|---|---|---|
| LT = Form B, HT = Form A | 16.63 [15.34*] | 38.1 | 163.3 |
| LT = Form E, HT = Form A | 17.15 [17.94*] | 39.2 | 163.9 |

LT = low temperature form
HT = high temperature form
*ΔH from heat of solution data

Forms B and E were found to be quite close in energy, whereas Form A was found to be higher in energy with respect to both Forms B and E. The rank order of stability correlates with true density data of the crystal forms as measured by helium pycnometry (Form B, 1.46±0.01 g/cm³; Form E, 1.42±0.01 g/cm³; Form A, 1.34±0.01 g/cm³.)

By definition, the free energy difference between crystal forms is zero at the transition temperature. The transition temperature given in Table 5 above was calculated according to equation II:

Tₜ=ΔH/ΔS (equation II)

The similar transition temperatures for Form E/Form A and Form B/Form A pairs are related to the narrow energy difference between Forms E and B. The similar free energies of Forms E and B make it difficult to ascertain which form is the more thermodynamically stable at ambient temperature. For example, the heat of solution and eutectic melting data suggest that Form E is more stable, whereas the DSC data would suggest that Form B is the more stable form based on transition energies.

### Example 7

### Moisture Sorption

Moisture sorption data were collected at 25° C. from 0% to 80% RH using a Surface Measurement Systems Dynamic Vapor Water Sorption analyzer. The equilibrium window was for a dm/dT of 0.0003 or a maximum time of 120 minutes.

The moisture sorption profile of parecoxib sodium Form A at 25° C. is shown in FIG. 4. Form A sorbed less than 1% moisture over a 0-60% RH range, but deliquesced above 60% RH.

The moisture sorption profiles of parecoxib sodium Forms B and E are shown in FIGS. 8 and 12 respectively. Both Forms B and E were found to be less hygroscopic than Form A, sorbing less than 1% moisture over the full 0-80% RH range tested.

What is claimed is:

1. Parecoxib sodium in a crystalline form that is substantially anhydrous and substantially nonsolvated.

2. The parecoxib sodium of claim 1 that is Form A as characterized at least by a powder x-ray diffraction pattern having at least two 2θ values selected from the group consisting of 5.6, 9.6, 11.0 and 14.5±0.2 degrees.

3. The parecoxib sodium of claim 1 that is Form A as characterized at least by a powder x-ray diffraction pattern substantially in accordance with FIG. 1.

4. The parecoxib sodium of claim 1 that is Form A as characterized at least by a Fourier-transform infrared spectrum substantially in accordance with FIG. 2.

5. The parecoxib sodium of claim 1 that is Form A as characterized at least by a differential scanning calorimetry thermogram substantially in accordance with FIG. 3.

6. The parecoxib sodium of claim 1 that is Form B as characterized at least by a powder x-ray diffraction pattern having at least two 2θ values selected from the group consisting of 4.2, 8.3, 12.4, 16.7, 17.5, 20.8 and 24.7±0.2 degrees.

7. The parecoxib sodium of claim 1 that is Form B as characterized at least by a powder x-ray diffraction pattern substantially in accordance with FIG. 5.

8. The parecoxib sodium of claim 1 that is Form B as characterized at least by a Fourier-transform infrared spectrum substantially in accordance with FIG. 6.

9. The parecoxib sodium of claim 1 that is Form B as characterized at least by a differential scanning calorimetry thermogram substantially in accordance with FIG. 7.

10. The parecoxib sodium of claim 1 that is Form E as characterized at least by a powder x-ray diffraction pattern having at least two 2θ values selected from the group consisting of 8.8, 11.3, 15.6, 22.4, 23.5 and 26.4±0.2 degrees.

11. The parecoxib sodium of claim 1 that is Form E as characterized at least by a powder x-ray diffraction pattern substantially in accordance with FIG. 9.

12. The parecoxib sodium of claim 1 that is Form E as characterized at least by a Fourier-transform infrared spectrum substantially in accordance with FIG. 10.

13. The parecoxib sodium of claim 1 that is Form E as characterized at least by a differential scanning calorimetry thermogram substantially in accordance with FIG. 11.

14. A parecoxib sodium drug substance comprising at least about 90% of said parecoxib sodium in one or more anhydrous, nonsolvated crystal forms.

15. The drug substance of claim 14 wherein at least about 95% of the parecoxib sodium is in one or more anhydrous, nonsolvated crystal forms.

16. The drug substance of claim 14 wherein substantially all of the parecoxib sodium is in one or more anhydrous, nonsolvated crystal forms.

17. The drug substance of claim 14 wherein said one or more anhydrous, nonsolvated crystal forms comprise Form A.

18. The drug substance of claim 14 wherein said one or more anhydrous, nonsolvated crystal forms comprise Form B.

19. The drug substance of claim 14 wherein said one or more anhydrous, nonsolvated crystal forms comprise Form E.

20. A process for preparing a parecoxib sodium drug substance having at least about 90% Form A, the process comprising the steps of (a) crystallizing parecoxib sodium from a crystallizing solvent to produce a crystalline form of parecoxib sodium, and (b) heating the resulting crystalline parecoxib sodium at a temperature of about 110° C. to about 230° C. to produce said drug substance.

21. A process for preparing a pharmaceutical composition useful in treatment of a COX-2 mediated disorder, the process comprising a step of dissolving in an aqueous medium the parecoxib sodium drug substance of claim 14, together with at least one pharmaceutically acceptable excipient, to form a solution.

22. The process of claim 21, further comprising a step of lyophilizing said solution to provide a solid particulate composition comprising amorphous parecoxib sodium.

23. A pharmaceutical composition comprising a therapeutically effective amount of the parecoxib sodium drug substance of claim 14 and at least one pharmaceutically acceptable excipient.

24. A method of treating a COX-2 mediated disorder in a subject, the method comprising administering to the subject a therapeutically effective amount of the composition of claim 23.

25. Use of the parecoxib sodium drug substance of claim 14 in manufacture of a medicament for treating a COX-2 mediated disorder in a subject.
